(19) United States
(12) Patent Application Publication
(10) Pub. No.: US 2013/0102918 A1
(43) Pub. Date: Apr. 25, 2013

(54) SYSTEM AND METHOD FOR DIAGNOSING AND TREATING PSYCHIATRIC DISORDERS

(71) Applicants: Amit Etkin, Los Altos, CA (US); Alan F. Schatzberg, Los Altos, CA (US); Anett Gyurak, Mountain View, CA (US)

(72) Inventors: Amit Etkin, Los Altos, CA (US); Alan F. Schatzberg, Los Altos, CA (US); Anett Gyurak, Mountain View, CA (US)

(21) Appl. No.: 13/586,641

(22) Filed: Aug. 15, 2012

Related U.S. Application Data
(60) Provisional application No. 61/524,146, filed on Aug. 16, 2011.

(51) Int. Cl. G09B 19/00; A61B 5/0476; A61B 5/00

(52) U.S. Cl. CPC G09B 19/00; A61B 5/4848; A61B 5/0476
USPC 600/544; 434/236; 600/558

(57) ABSTRACT

Systems and methods for diagnosing and treating psychiatric disorders are provided. For example, in one embodiment, the systems and methods generally include: (a) presenting an emotional conflict task to a patient; (b) receiving an input from the patient in response to the emotional conflict task; (c) assessing the patient's response to the emotional conflict task; and (d) modifying the emotional conflict task based on the patient's response. Such systems and methods may also be employed in a computerized training system for treating a patient with, or at risk of, a psychiatric disorder by training the patient's implicit emotional regulation.

### CROSS-REFERENCE

This application claims the benefit of U.S. Provisional Patent Application No. 61/524,146, filed Aug. 16, 2011, which application is incorporated herein by reference in its entirety.

### STATEMENT REGARDING FEDERALLY SPONSORED RESEARCH

This invention was made with government support under Grant No. P30 MH089888 awarded by the National Institute of Mental Health. The government has certain rights in the invention.

### SUMMARY

Disclosed herein are systems and methods for diagnosing and treating psychiatric disorders. For example, in one embodiment, the systems and methods generally include: (a) presenting an emotional conflict task to a patient; (b) receiving an input from the patient in response to the emotional conflict task; (c) assessing the patient's response to the emotional conflict task; and (d) modifying the emotional conflict task based on the patient's response. Such systems and methods may also be employed in a computerized training system for treating a patient with, or at risk of, a psychiatric disorder by training the patient's implicit emotional regulation.

### BRIEF DESCRIPTION OF THE FIGURES

The accompanying drawings, which are incorporated herein, form part of the specification. Together with this written description, the drawings further serve to explain the principles of, and to enable a person skilled in the relevant art(s), to make and use the claimed systems and methods.

FIG. 1 is a high-level flow chart outlining one embodiment of the present invention.

FIG. 2 is a screen shot of an exemplary emotional conflict task.

FIG. 3 is a schematic drawing of a computer system used to implement the methods.

FIGS. 4A-C depict adaptation to emotional conflict on an emotional conflict task in patient and healthy comparison groups.

FIGS. 5A and 5B depict brain activation in the ventral cingulate and the amygdala in the contrast examining emotional conflict adaptation during incongruent trials in an emotional conflict task in patient and healthy comparison groups.

FIGS. 6A and 6B depict ventral cingulate-amygdala functional connectivity and activation correlations in patient and healthy comparison groups.

FIGS. 7A-C depict correlation between postincongruent incongruent trial minus postcongruent incongruent trial reaction time difference scores and brain activation for the same contrast.

### DETAILED DESCRIPTION

The present disclosure generally relates to systems and methods for diagnosing and treating psychiatric disorders, and to a biomarker for anxiety disorders.

There is growing evidence suggesting abnormalities in the processing and regulation of emotion, in a wide range of psychopathology, using behavioral and neuroimaging tools. Etkin et al. (Am J Psych 2010, Etkin & Schatzberg, Am J Psych 2011) demonstrated that patients with anxiety and depression have brain activation and behavioral abnormalities during a specific and objectively-assessable type of emotion regulation. Such emotion regulation has been termed implicit (i.e., non-conscious) emotion regulation.

### Training Game

In one embodiment presented, there is provided a neurobehavioral intervention that serves as a behavioral probe for an adaptive, computerized training game, which can enhance the emotion regulatory process tapped into by an emotional conflict task. During the neurobehavioral training game, subjects are told to focus on identifying the expression of a face (e.g., fear, happy, disgust, etc.) that shows a canonical (strong) version of that expression. Facial expressions are presented at random with a word (e.g., “fear,” “happy,” “disgust,” in text form) overlaying the image. Patients are told to ignore the word overlaid on the face, which are read involuntarily and create an emotional interference with the emotion shown on the face when the face and word are not the same (i.e., are an incongruent pair). Subjects doing the training task are further told that when they see, at the beginning of the training course, a fixation cross come up in one color (e.g., red) rather than another (e.g., black), this will indicate that the next stimulus is an incongruent stimulus and that they should focus hard to ignore the word and identify the expression accurately. Warning (e.g., red) fixation crosses appear initially before 90% of the incongruent stimuli, and then decrease each training day, until they do not appear at all. In this way, subjects render more automatic, through practice, the emotion regulatory strategy involved in focusing on expression identification while ignoring the emotionally conflicting word. As such, patients are progressively weaned off the predictive cue, and have to be able to non-consciously implement the regulation strategy consistently without a cue. Further, the time available for the patient to respond to a stimulus is continually titrated to maintain an ˜70% accuracy throughout the training game. One goal is to make the training game continually challenging, even as patients learn to better perform the emotional conflict task.

The results of this training, tested in 17 healthy individuals, using an ˜25 min/day training period over 10 days (excluding weekends) showed that these individuals improved in overall reaction times during training (average speed of correct responses getting faster), and showed a reduction in the magnitude of the reaction time slowdown associated with emotional conflict. Further, when comparing pre- vs post-training performance on the two “benchmark tests”—the emotional conflict task and an analogous non-emotional conflict task that engages different circuitry but has the same overall structure and behavioral output—it was found that implicit emotion regulation was improved after training, while implicit cognitive (non-emotional) regulation was unchanged. Individuals with the worse baseline implicit emotion regulation performance on reaction times were those who saw the greatest benefit with training, supporting the utility of this approach for individuals with deficits in this emotional regulatory process (low resilient individuals, or those experiencing anxiety or depression symptoms).

Within a training session, the majority of the reduction in the magnitude of the emotional conflict reaction time effect was achieved within the first 3-5 minutes of training. Across days, the majority of the overall benefit was reached within 5 days. This suggests that brief (3-5 minutes) daily training may be sufficient, even over a short course, to yield benefits.

The following detailed description of the figures refers to the accompanying drawings that illustrate exemplary embodiments. Other embodiments are possible. Modifications may be made to the embodiments described herein without departing from the spirit and scope of the present invention. Therefore, the following detailed description is not meant to be limiting.

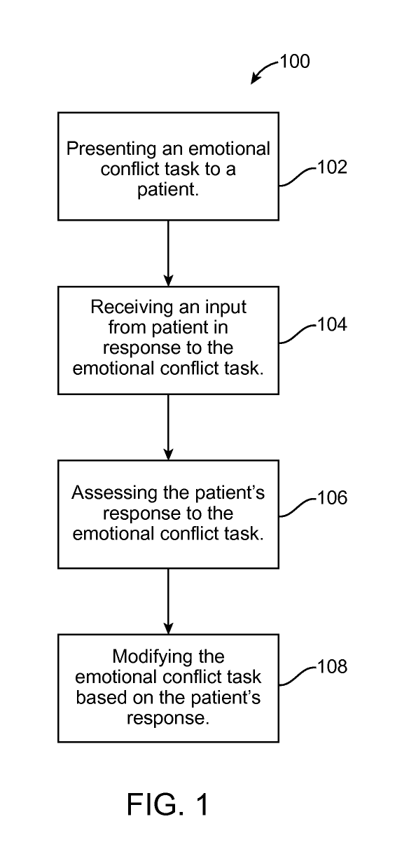

FIG. 1 is a high-level flow chart outlining one embodiment of the present invention. For example, presented is a method for treating a patient with, or at risk of, a psychiatric disorder by training the patient's implicit emotional regulation. The training includes presenting an emotional conflict task to a patient, in step 102. The emotional conflict task may include presenting a series of stimuli-pairs, each stimuli-pair representing at least one emotional significance. The emotional significance may be selected from the group consisting of: happiness, disgust, fear, sadness, surprise, anger, an idiosyncratic emotion, and any equivalents thereof.

In one embodiment, at least one of the stimuli-pairs may include two incongruent stimuli (I). Additionally, at least one of the stimuli-pairs may include two congruent stimuli (C). The series of stimuli-pairs may include a sequence of stimuli-pairs selected from the group consisting of: an incongruent stimuli-pair preceded by another incongruent stimuli-pair (iI); an incongruent stimuli-pair preceded by a congruent stimuli-pair (cI); a congruent stimuli-pair preceded by another congruent stimuli-pair (cC); and a congruent stimuli-pair preceded by an incongruent stimuli-pair (iC).

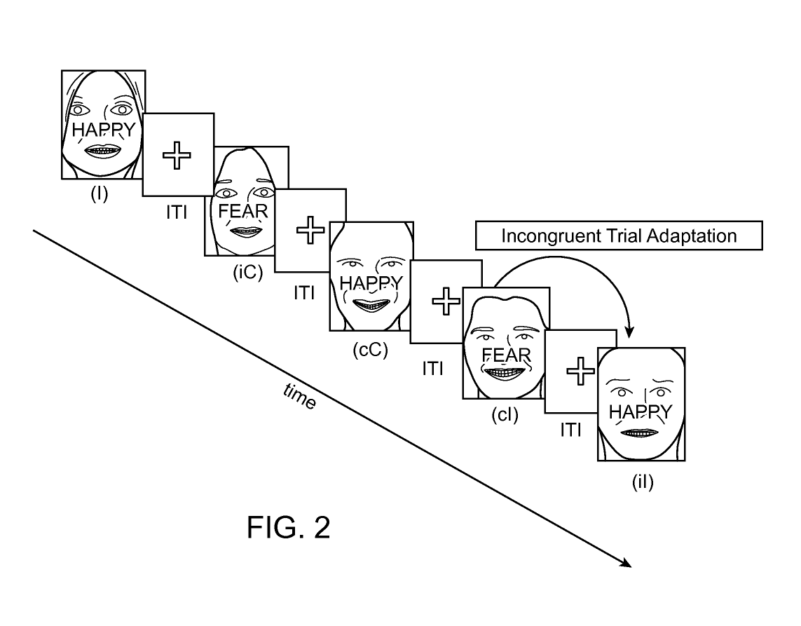

FIG. 2 is a screen shot of an exemplary emotional conflict task. In the embodiment shown in FIG. 2, each stimuli-pair includes an emotional face with an emotional label overlaying the emotional face. In alternative embodiments, a stimuli-pair may include any image and an overlaying word, or two images without text. A series of fixation crosses (ITI) are displayed between stimuli-pairs. FIG. 2 shows a series of stimuli-pairs sequenced as follows: I; ITI; iC; ITI; cC; ITI; cI; ITI; and iI.

Referring back to FIG. 1, an input is then received from the patient in response to the emotional conflict task, in step 104. For example, the patient may be asked to identify the emotion shown on the face will ignoring the word overlaid on the face. Such request forces the patient to deal with an involuntarily read of the word, which creates an emotional interference with the emotion shown on the face when the face and word are not the same (i.e., the face and the word are an incongruent pair). The patient's input may be provided by any known computerized input means.

The patient's response to the emotional conflict task is then assessed, in step 106. For example, a time between when the patient is presented with the stimuli-pair and when the input is received from the patient may be measured to assess the patient's response. The patient's ability to correctly identify the emotional significance of the stimuli-pair may also be assessed. Alternatively, both the time between when the patient is subjected to a stimuli-pair and when the patient correctly identifies the emotional significance of the stimuli-pair may be assessed.

Finally, the emotional conflict task is modified based on the patient's response, in step 108. For example, how a subsequent stimuli-pair is presented to the patient may be modified based on the amount of time between when the patient is presented with the stimuli-pair and when the input is received from the patient. Alternatively, how a subsequent stimuli-pair is presented to the patient may be modified based on the amount of time between when the patient is presented with the stimuli-pair and when the patient correctly identifies the emotional significance of the stimuli-pair. When fixation crosses are shown between stimuli-pairs, the amount of time in which a fixation cross is presented to the patient may be changed based on the patient's response or how long the patient has been using the training system.

In one embodiment, for example, the modification of subsequent stimuli-pair may be performed according to the following protocol:

(1) if the patient fails to correctly identify the emotional significance of a preceding stimuli-pair, a subsequent stimuli-pair is presented to the patient for a length of time between 63-73 ms greater than the length of time that the preceding stimuli-pair was presented to the patient;

(2) if the patient correctly identifies the emotional significance of a preceding stimuli-pair, a subsequent stimuli-pair is presented to the patient for a length of time between 46-56 ms greater than the length of time that the preceding stimuli-pair was presented to the patient;

(3) if the patient correctly identifies the emotional significance of two preceding stimuli-pairs, a subsequent stimuli-pair is presented to the patient for a length of time between 29-39 ms greater than the length of time that the preceding stimuli-pair was presented to the patient;

(4) if the patient correctly identifies the emotional significance of three preceding stimuli-pairs, a subsequent stimuli-pair is presented to the patient for a length of time between 12-22 ms greater than the length of time that the preceding stimuli-pair was presented to the patient;

(5) if the patient correctly identifies the emotional significance of four preceding stimuli-pairs, a subsequent stimuli-pair is presented to the patient for a length of time about equal to the length of time that the preceding stimuli-pair was presented to the patient; and/or

(6) if the patient correctly identifies the emotional significance of five preceding stimuli-pairs, a subsequent stimuli-pair is presented to the patient for a length of time between 12-22 ms less than the length of time that the preceding stimuli-pair was presented to the patient.

In one embodiment, the patient is subject to the emotional conflict task for less than 10 minutes. In another embodiment, the patient is subject to the emotional conflict task for less than 4 minutes.

### Additional Embodiments

In another embodiment, there is provided a method for treating a patient with, or at risk of, a psychiatric disorder by training the patient's implicit emotional regulation.

The method includes: (a) presenting an emotional conflict task to the patient, wherein the emotional conflict task includes presenting a series of stimuli-pairs, each stimuli-pair representing at least one emotional significance; (b) receiving an input from the patient in response to one of the stimuli-pair; (c) measuring a time between when the patient is presented with the stimuli-pair and when the input is received from the patient; and (d) modifying how a subsequent stimuli-pair is presented to the patient based on the amount of time between when the patient is presented with the stimuli-pair and when the input is received from the patient. In one embodiment, said method is performed on a computerized system.

In another embodiment, there is provided a system comprising: (a) means for presenting an emotional conflict task to the patient, wherein the emotional conflict task includes presenting a series of stimuli-pairs, each stimuli-pair representing at least one emotional significance; (b) means for receiving an input from the patient in response to one of the stimuli-pair; (c) means for measuring a time between when the patient is presented with the stimuli-pair and when the input is received from the patient; and (d) means for modifying how a subsequent stimuli-pair is presented to the patient based on the amount of time between when the patient is presented with the stimuli-pair and when the input is received from the patient. In one embodiment, said method is performed on a computerized system.

In still another embodiment, there is provided a method for treating a patient with, or at risk of, a psychiatric disorder by training the patient's implicit emotional regulation. The method includes: (a) presenting an emotional conflict task to the patient, wherein the emotional conflict task includes presenting a series of stimuli-pairs, each stimuli-pair representing at least one emotional significance; (b) receiving an input from the patient in response to one of the stimuli-pair; (c) assessing the ability of the patient to correctly identify the emotional significance of the stimuli-pair; and (d) modifying how a subsequent stimuli-pair is presented to the patient based on whether the patient correctly identified the emotional significance of a preceding stimuli-pair. In one embodiment, said method is performed on a computerized system.

In yet another embodiment, there is provided a system comprising: (a) means for presenting an emotional conflict task to the patient, wherein the emotional conflict task includes presenting a series of stimuli-pairs, each stimuli-pair representing at least one emotional significance; (b) means for receiving an input from the patient in response to one of the stimuli-pair; (c) means for assessing the ability of the patient to correctly identify the emotional significance of the stimuli-pair; and (d) means for modifying how a subsequent stimuli-pair is presented to the patient based on whether the patient correctly identified the emotional significance of a preceding stimuli-pair. In one embodiment, said method is performed on a computerized system.

### Communication Between Parties

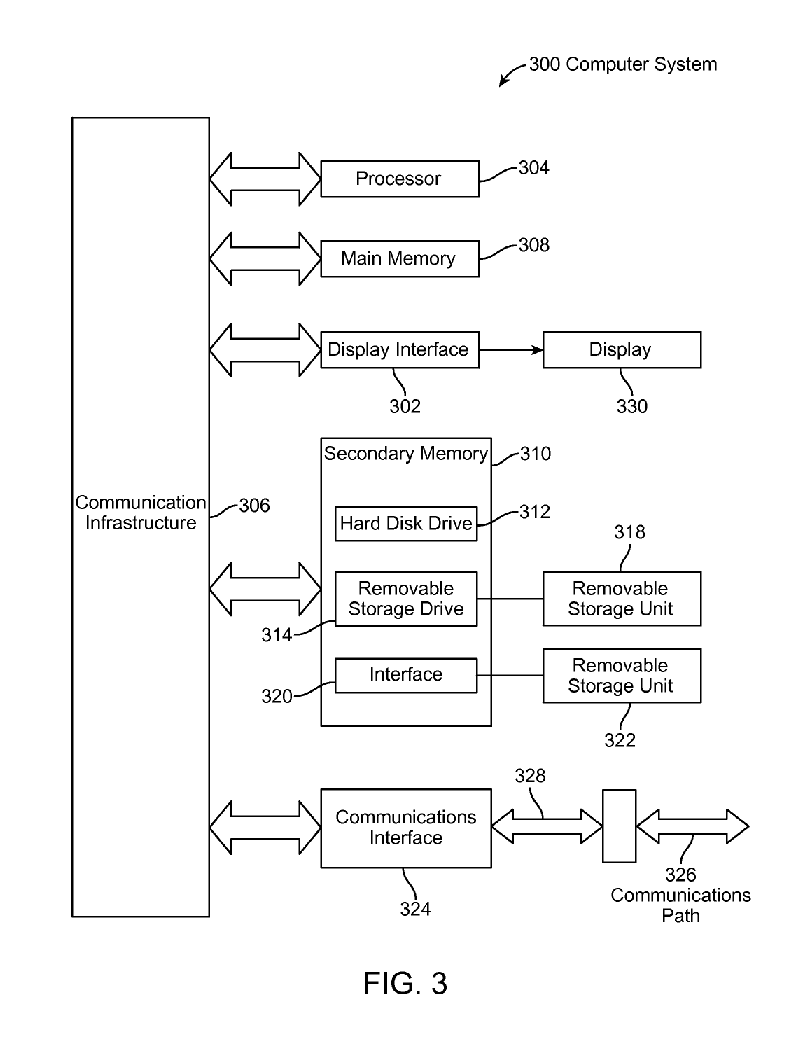

In one embodiment, communication between the various parties and components of the present disclosure is accomplished over a network consisting of electronic devices connected either physically or wirelessly, wherein digital information is transmitted from one device to another. Such devices (e.g., end-user devices and/or servers) may include, but are not limited to: a desktop computer, a laptop computer, a handheld device or PDA, a cellular telephone, a set top box, an Internet appliance, an Internet TV system, a mobile device or tablet, or systems equivalent thereto. Exemplary networks include a Local Area Network, a Wide Area Network, an organizational intranet, the Internet, or networks equivalent thereto. The functionality and system components of an exemplary computer and network are further explained in conjunction with FIG. 3, below.

### Computer Implementation

In one embodiment, the present disclosure provides one or more computer systems capable of carrying out the functionality described herein. For example, FIG. 3 is a schematic drawing of a computer system 300 used to implement the methods presented above. Computer system 300 includes one or more processors, such as processor 304. The processor 304 is connected to a communication infrastructure 306 (e.g., a communications bus, cross-over bar, or network). Computer system 300 can include a display interface 302 that forwards graphics, text, and other data from the communication infrastructure 306 (or from a frame buffer not shown) for display on a local or remote display unit 330.

Computer system 300 also includes a main memory 308, such as random access memory (RAM), and may also include a secondary memory 310. The secondary memory 310 may include, for example, a hard disk drive 312 and/or a removable storage drive 314, representing a floppy disk drive, a magnetic tape drive, an optical disk drive, flash memory device, etc. The removable storage drive 314 reads from and/or writes to a removable storage unit 318. Removable storage unit 318 represents a floppy disk, magnetic tape, optical disk, flash memory device, etc., which is read by and written to by removable storage drive 314. As will be appreciated, the removable storage unit 318 includes a computer usable storage medium having stored therein computer software, instructions, and/or data.

In alternative embodiments, secondary memory 310 may include other similar devices for allowing computer programs or other instructions to be loaded into computer system 300. Such devices may include, for example, a removable storage unit 322 and an interface 320. Examples of such may include a program cartridge and cartridge interface (such as that found in video game devices), a removable memory chip (such as an erasable programmable read only memory (EPROM), or programmable read only memory (PROM)) and associated socket, and other removable storage units 322 and interfaces 320, which allow computer software, instructions, and/or data to be transferred from the removable storage unit 322 to computer system 300.

Computer system 300 may also include a communications interface 324. Communications interface 324 allows computer software, instructions, and/or data to be transferred between computer system 300 and external devices. Examples of communications interface 324 may include a modem, a network interface (such as an Ethernet card), a communications port, a Personal Computer Memory Card International Association (PCMCIA) slot and card, etc. Software and data transferred via communications interface 324 are in the form of signals 328 which may be electronic, electromagnetic, optical or other signals capable of being received by communications interface 324. These signals 328 are provided to communications interface 324 via a communications path (e.g., channel) 326. This channel 326 carries signals 328 and may be implemented using wire or cable, fiber optics, a telephone line, a cellular link, a radio frequency (RF) link, a wireless communication link, and other communications channels.

In this document, the terms “computer-readable storage medium,” “computer program medium,” and “computer usable medium” are used to generally refer to media such as removable storage drive 314, removable storage units 318, 322, data transmitted via communications interface 324, and/or a hard disk installed in hard disk drive 312. These computer program products provide computer software, instructions, and/or data to computer system 300. These computer program products also serve to transform a general purpose computer into a special purpose computer programmed to perform particular functions, pursuant to instructions from the computer program products/software. Embodiments of the present invention are directed to such computer program products.

Computer programs (also referred to as computer control logic) are stored in main memory 308 and/or secondary memory 310. Computer programs may also be received via communications interface 324. Such computer programs, when executed, enable the computer system 300 to perform the features of the present invention, as discussed herein. In particular, the computer programs, when executed, enable the processor 304 to perform the features of the presented methods. Accordingly, such computer programs represent controllers of the computer system 300. Where appropriate, the processor 304, associated components, and equivalent systems and sub-systems thus serve as “means for” performing selected operations and functions. Such “means for” performing selected operations and functions also serve to transform a general purpose computer into a special purpose computer programmed to perform said selected operations and functions.

In an embodiment where the invention is implemented using software, the software may be stored in a computer program product and loaded into computer system 300 using removable storage drive 314, interface 320, hard drive 312, communications interface 324, or equivalents thereof. The control logic (software), when executed by the processor 304, causes the processor 304 to perform the functions and methods described herein.

In another embodiment, the methods are implemented primarily in hardware using, for example, hardware components such as application specific integrated circuits (ASICs) Implementation of the hardware state machine so as to perform the functions and methods described herein will be apparent to persons skilled in the relevant art(s). In yet another embodiment, the methods are implemented using a combination of both hardware and software.

Embodiments of the invention may also be implemented as instructions stored on a machine-readable medium, which may be read and executed by one or more processors. A machine-readable medium may include any mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact result from computing devices, processors, controllers, or other devices executing firmware, software, routines, instructions, etc.

For example, in one embodiment, there is provided a computer-readable storage medium used for treating a patient with, or at risk of, a psychiatric disorder by training the patient's implicit emotional regulation. The computer-readable storage medium includes instructions executable by at least one processing device that, when executed, cause the processing device to: (a) present an emotional conflict task to the patient, wherein the emotional conflict task includes presenting a series of stimuli-pairs, each stimuli-pair representing at least one emotional significance; (b) receive an input from the patient in response to the stimuli-pair; (c) measure a time between when the patient is presented with the stimuli-pair and when the input is received from the patient; and (d) modify how a subsequent stimuli-pair is presented to the patient based on the amount of time between when the patient is presented with the stimuli-pair and when the input is received from the patient. The computer-readable storage medium may further include instructions executable by at least one processing device that, when executed, cause the processing device to: (e) assess the ability of the patient to correctly identify the emotional significance of the stimuli-pair; (f) measure a time between when the patient is subjected to a stimuli-pair and when the patient correctly identifies the emotional significance of the stimuli-pair; and/or (g) modify how a subsequent stimuli-pair is presented to the patient based on the amount of time between when the patient is presented with the stimuli-pair and when the patient correctly identifies the emotional significance of the stimuli-pair.

In one embodiment, at least one of the stimuli-pairs may include two incongruent stimuli. Additionally, at least one of the stimuli-pairs may include two congruent stimuli. The series of stimuli-pairs may include a sequence of stimuli-pairs selected from the group consisting of: an incongruent stimuli-pair preceded by another incongruent stimuli-pair, an incongruent stimuli-pair preceded by a congruent stimuli-pair, a congruent stimuli-pair preceded by another congruent stimuli-pair, and a congruent stimuli-pair preceded by an incongruent stimuli-pair.

In one embodiment, at least one stimuli-pair may include an image and an overlaying word. Alternatively, at least one stimuli-pair may include two images. Further, at least one a stimuli-pair may include an emotional face with an emotional label overlaying the emotional face.

In one embodiment, the emotional conflict task may include a series of fixation crosses displayed between stimuli-pairs. The computer-readable storage medium may further include instructions executable by at least one processing device that, when executed, cause the processing device to change the amount of time in which a fixation cross is presented to the patient based on how long the patient has used the computer-readable storage medium.

In one embodiment, the patient is subject to the emotional conflict task for less than 10 minutes. In another embodiment, the patient is subject to the emotional conflict task for less than 4 minutes. The series of emotional significance may be selected from the group consisting of: happiness, disgust, fear, sadness, surprise, anger, an idiosyncratic emotion, and equivalents thereof.

In another embodiment, there is provided a computer-readable storage medium used for treating a patient with, or at risk of, a psychiatric disorder by training the patient's implicit emotional regulation. The computer-readable storage medium includes instructions executable by at least one processing device that, when executed, cause the processing device to: (a) present an emotional conflict task to the patient, wherein the emotional conflict task includes presenting a series of stimuli-pairs, each stimuli-pair representing at least one emotional significance; (b) receive an input from the patient in response to the stimuli-pair; (c) assess the ability of the patient to correctly identify the emotional significance of the stimuli-pair; and (d) modify how a subsequent stimuli-pair is presented to the patient based on whether the patient correctly identified the emotional significance of a preceding stimuli-pair.

The modification of subsequent stimuli-pair may be performed according to the following protocol: (1) if the patient fails to correctly identify the emotional significance of a preceding stimuli-pair, a subsequent stimuli-pair is presented to the patient for a length of time between 63-73 ms greater than the length of time that the preceding stimuli-pair was presented to the patient; (2) if the patient correctly identifies the emotional significance of a preceding stimuli-pair, a subsequent stimuli-pair is presented to the patient for a length of time between 46-56 ms greater than the length of time that the preceding stimuli-pair was presented to the patient; (3) if the patient correctly identifies the emotional significance of two preceding stimuli-pairs, a subsequent stimuli-pair is presented to the patient for a length of time between 29-39 ms greater than the length of time that the preceding stimuli-pair was presented to the patient; (4) if the patient correctly identifies the emotional significance of three preceding stimuli-pairs, a subsequent stimuli-pair is presented to the patient for a length of time between 12-22 ms greater than the length of time that the preceding stimuli-pair was presented to the patient; (5) if the patient correctly identifies the emotional significance of four preceding stimuli-pairs, a subsequent stimuli-pair is presented to the patient for a length of time about equal to the length of time that the preceding stimuli-pair was presented to the patient; and/or (6) if the patient correctly identifies the emotional significance of five preceding stimuli-pairs, a subsequent stimuli-pair is presented to the patient for a length of time between 12-22 ms less than the length of time that the preceding stimuli-pair was presented to the patient.

### Anxiety Biomarker

The present disclosure provides a method of assessing the effectiveness of a treatment (e.g., an experimental treatment) for a psychiatric disorder in an individual, where the psychiatric disorder is an anxiety disorder or a depressive disorder; and a method for predicting the response of an individual to treatment (e.g., an experimental treatment) for a psychiatric disorder in an individual, where the psychiatric disorder is an anxiety disorder or a depressive disorder. The methods rely upon use of the emotional conflict task as a “biomarker” for anxiety, and allow one to discriminate between an effect of a drug or other treatment (e.g., an experimental drug treatment) on anxiety or a depressive disorder. The emotional conflict task, and an individual's response thereto, can be used as a biomarker for anxiety, including in the context of depression. Depression without anxiety can be distinguished from depression with anxiety using this biomarker, thereby allowing the use of this biomarker in drug development, e.g., for the development of drugs to treat anxiety per se, even when other psychiatric conditions (such depression) are present.

The methods generally involve assessing the ability of an individual to implicitly regulate emotional conflict by subjecting the individual to an emotional conflict task. The ability of the individual to implicitly regulate emotional conflict provides an indication as to whether the treatment is effective to treat an anxiety disorder. Failure to implicitly regulate emotional conflict following treatment with a particular drug treatment for anxiety (e.g., an experimental drug for treating anxiety disorder) indicates a reduced likelihood that the individual will exhibit a beneficial clinical response to the drug. The ability (or improved ability) to implicitly regulate emotional conflict following treatment with a particular drug treatment for anxiety (e.g., an experimental drug for treating anxiety disorder) indicates an increased likelihood that the individual will exhibit a beneficial clinical response to the drug. The ability to implicitly regulate emotional conflict is measured by reaction time adaptation to emotionally incongruent trials in an emotional conflict task.

The emotional conflict task is as described above, and in Example 1. Healthy individuals (e.g., “control” individuals who have been determined not to have an anxiety disorder or a depressive disorder) can be subjected to the emotional conflict task, to provide a normal control value, or range of normal control values. Such individuals may exhibit an improvement in implicit emotional regulation. However, an individual who has an anxiety disorder, and who exhibits a beneficial clinical response to a drug treatment for anxiety (e.g., an experimental drug for treating anxiety disorder) will exhibit a much greater improvement in implicit emotional regulation, as measured by the emotional conflict task, compared to a normal control.

In addition to subjecting an individual to an emotional conflict task, an individual can further be subjected to various tests for brain activity. For example, the individual can further be tested by detecting activation in limbic and prefrontal brain regions; activation in such brain regions can provide further indication as to the effectiveness of the treatment.

A drug whose effectiveness in treating an anxiety disorder is being tested is administered to one or more individuals (e.g., “test individuals”); and the test individuals are subjected to the emotional conflict test. A test drug that provides for an improvement in response to the emotional conflict task (e.g., an improvement in implicit emotional regulation) is considered a candidate for further development, for use in treating an anxiety disorder.

The emotional conflict task can be administered to a test individual before drug treatment (e.g., before treatment with an experimental drug for treating an anxiety disorder), to provide a reference response value. For example, where an individual who has an anxiety disorder exhibits a reduced reaction time adaptation to emotionally incongruent trials in response to a drug (e.g., an experimental drug for treating an anxiety disorder), compared to the reaction time adaptation in the absence of treatment with the drug (e.g., before treatment with the drug), that drug is considered a candidate for treating an anxiety disorder. As an example, the emotional conflict task is administered to a test individual before drug treatment (e.g., before treatment with an experimental drug for treating an anxiety disorder), to provide a reference or “pre-treatment” reaction time adaptation value. A drug (e.g., an experimental drug for treating an anxiety disorder) is then administered to the test individual, and the test individual is subjected to an emotional conflict task. If the test individual exhibits a substantially reduced reaction time adaptation (e.g., reaction time adaptation is reduced by at least about 10%, at least about 15%, at least about 20%, at least about 25%, at least about 30%, at least about 35%, at least about 40%, at least about 45%, at least about 50%, or more than 50%), compared to the reference or pre-treatment reaction time adaptation value, the drug is considered a candidate for treating an anxiety disorder.

Test individuals can include those who meet DSM-IV criteria for an anxiety disorder, e.g., a generalized anxiety disorder. Exclusion criteria can include those listed in Example 1, below. In general, test individuals are not receiving regular psychiatric medications. In some instances, a test individual has both anxiety and at least a second psychiatric disorder, such as a depressive disorder. In some instances, a test individual has anxiety, and no other psychiatric disorders.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); ms or msec, millisecond(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Application of an Emotional Conflict Task to Patients with Anxiety Disorders and to Patients with Depressive Disorders

Summary:

The data presented in this Example support the existence of a common anxiety/depression ventral cingulate-amygdalar abnormality, which may relate to a shared genetic etiology. Compensatory engagement of cognitive control circuitry in depression illustrates how the complex nature of psychopathology arises from the interaction of deficits and compensation, all of which can occur at an implicit level.

In this study, implicit regulation of emotional processing was examined in an emotional conflict task in patients with generalized anxiety disorder, depression or both, in order to test, at the behavioral and neural level, contrasting conceptualizations of these disorders. At the behavioral data, evidence was found supporting the “independent factor” model, whereby anxiety and depression reflect separate and dissociable processes. That is, failure to implicitly regulate emotional conflict, indexed through reaction time adaptation to emotionally incongruent trials, was perturbed in anxiety, and not depression.

### Methods

### Participants

A total of 89 subjects participated in this study, after providing informed consent. Current-episode DSM-IV-based psychiatric diagnoses (33) were determined through both an informal clinical interview, and the MINI structured diagnostic interview (34, 35). All subject screening and diagnosis was carried out by a single rater (AE), who is a trained psychiatrist, applying consistent diagnostic criteria for differentiating between the four groups examined. Exclusion criteria were bipolar, psychotic, substance abuse or post-traumatic stress disorders, a history of a neurological disorder, head trauma or loss of consciousness, claustrophobia or regular use of benzodiazepines, opiate or thyroid medications. No patient was taking regular psychiatric medications (e.g. antidepressants). If they used “as needed” benzodiazepines, none took them within 48 hours of the scan. The “anxiety only” (N=18) and “depression only” (N=14) groups consisted of patients in which generalized anxiety disorder or major depressive disorder, respectively, were the primary diagnosis, without comorbidity with the other, while the “comorbid” group (N=25) consisted of patients with comorbid generalized anxiety and major depression. Comorbidities with other disorders are noted in table 1. All controls (N=32) were free of any current or past Axis I conditions or psychiatric medications. All participants completed the questionnaires noted in table 1 (36-41). Emotional conflict data on 24 of the healthy controls and 17 of the anxiety only patients was reported in our previous study (23).

TABLE 1
Demographic characteristics and clinical measures for healthy
comparison subjects and patients with generalized anxiety disorder only, major
depression only, or comorbid anxiety and depression

| | | | Anxiety- | | Depression- | | | |
|---|---|---|---|---|---|---|---|---|
| | Comparison | | Only | | Only | | Comorbid | |
| | Group | | Group [symbol] | | Group [symbol] | | Group^{c} | |
| | N | % | N | % | N | % | N | % |
| Female | 23 | 72 | 11 | 61 | 10 | 71 | 17 | 68 |
| | Mean | SD | Mean | SD | Mean | SD | Mean | SD |
| Age (years) | 35.6 | 11.1 | 31.3 | 9.5 | 32.2 | 11.7 | 33 | 10 |
| Education (years) | 17 | 2 | 16.5 | 2.2 | 15.4 | 2.1 | 16.2 | 2.5 |
| State-Trait Anxiety Inventory (36), trait anxiety | 30.4 | 6.1 | 51.9 | 8.1 | 58 | 11.2 | 62.8 | 8.7 |
| score | | | | | | | | |
| Fenn State Worry Questionnaire score (37) | 32.9 | 8.9 | 61.4 | 8.6 | 53.5 | 10.9 | 61.2 | 11.5 |
| Beck Anxiety Inventory score (38) | 3.6 | 3.7 | 22.7 | 11.7 | 14.4 | 6.6 | 36 | 12 |
| Beck Depression Inventory score (39) | 3.1 | 3.3 | 14.6 | 8.9 | 27.6 | 8.7 | 32 | 8.2 |
| Mood and Anxiety Symptom Questionnaire | | | | | | | | |
| Anxious arousal subscore (40) | 18.1 | 1.7 | 25.9 | 7.4 | 23.2 | 3.5 | 33 | 11.6 |
| Anhedonic depression subscore (41) | 48.1 | 10 | 69.4 | 12.4 | 87.1 | 11.1 | 88 | 7.7 |

^{a}Nine had no comorbid disorders, five had one comorbid disorder (two with dysthymia and three with social anxiety), three had two comorbid disorders (two with social anxiety and panic disorder and one with social anxiety and obsessive-compulsive disorder).
^{b}Thirteen had no comorbid disorders, and one had comorbid bulimia nervosa.
^{c}Eighteen had no comorbid disorders, six had one comorbid disorder (four with social anxiety and two with panic disorder), and one had two comorbid disorders (obsessive-compulsive disorder and bulimia nervosa).
^{d}All factors, interaction. [symbol]
All factors. [symbol]
indicates data missing or illegible when filed

### Experimental Paradigm

The emotional conflict task was performed as previously described (21-23), and consisted of 148 presentations of happy or fearful facial expression photographs (42), overlaid with the words “FEAR” or “HAPPY”. Stimuli were presented for 1000 ms, with a varying inter-stimulus interval of 3000-5000 ms (mean 4000 ms), in a pseudo-random order, counterbalanced across trial types for expression, word, response button and gender. Subjects indicated facial affect with a button press response.

fMRI Data Acquisition

Images were acquired on a 3T GE Signa scanner using a custom-built head coil. 29 axial slices (4.0 mm thickness with 0.5 mm gap) were acquired across the whole brain using a T2*weighted gradient echo spiral pulse sequence (TR=2000 msec, TE=30 msec, flip angle=80°, 1 interleave, FOV 22 cm, 64×64 matrix) (43). A high resolution T1 weighted spoiled grass gradient recalled (SPGR) inversion recovery 3D MRI sequence was used with the following parameters: TI=300 msec, TR=8 msec; TE=3.6 msec; flip angle=15°; 22 cm field of view; 124 slices in coronal plane; 256×192 matrix; 2 NEX, acquired resolution=1.5×0.9×1.1 mm

### Data Analysis

Functional MRI data were preprocessed using SPM5 software (http://www(dot)fil(dot)ion(dot)ucl(dot)ac(dot).uk/spm) implemented in Matlab (Mathworks, Inc., Natick, Mass.). Images were realigned to correct for motion, slice timing-corrected, spatially transformed to the Montreal Neurologic Institute coordinate system (44), resampled every 2 mm and smoothed with a 6 mm full-width half-maximum (FWHM) Gaussian kernel. During preprocessing, the effects of global signal were also removed separately for each voxel (45). Separate regressors for the stimulus events (convolved with a canonical HRF) were created for post-congruent incongruent trials, post-incongruent incongruent trials, post-congruent congruent trials and post-incongruent congruent trials, with error and post-error trials modeled separately. Additional regressors-of-no-interest corresponding to the six motion parameters were also included.

Results from first-level models (46) were submitted to group-level random-effects analyses. Groups were modeled in a 2×2 ANOVA using the Generalized Linear Model, with two across-subject factors corresponding to presence of the diagnosis of generalized anxiety or major depressive disorders (columns in the design matrix representing healthy, depression-only, anxiety-only and comorbid groups). To test the “independent factor” model, we created contrasts reflecting the effects of anxiety (anxiety-only and comorbid>healthy and depression-only; [−1, −1, 1, 1]), or depression (depression-only and comorbid>healthy and anxiety-only; [−1, 1, −1, 1]). The same group analysis was used to test the “common disorder” model, where we contrasted healthy subjects with all patient groups ([1, −⅓, −⅓, −⅓]). Behavioral and extracted brain activation data were analyzed in a similar fashion using SPSS (SPSS, Inc., Chicago, Ill.). We chose not to analyze all groups within a single 4-level group factor, as the variance associated with diagnoses for the comorbid group is overlapping with that in the anxiety-only and depression-only group (and hence not well-described in a 4-level group factor). Moreover, subject selection across the four groups was explicitly made with respect to the two main effect factors, and each subject could be uniquely identified by a combination of these factors.

Finally, for the analysis of adaptation, we analyzed effects of previous trial separately for current incongruent and congruent trials, since: 1) processing during congruent, but not incongruent, trials is potentially and variably confounded by subjects switching from labeling faces to labeling words, 2) separate behavioral work in the lab manipulating conflict adaptation has found dissociable effects on incongruent versus congruent trial adaptation (AE, unpublished observations), 3) brain activation is different between these forms of adaptation (AE, unpublished observations), and 4) we previously found deficits in generalized anxiety disorder selectively in incongruent trial adaptation (23).

For the psychophysiologic interaction analyses (47), we extracted for each subject, a deconvolved time-course from the ventral cingulate and amygdala clusters defined by the group-level regulation-related post-incongruent incongruent trial>post-congruent incongruent trial contrast and the evaluation-related post-congruent incongruent trial>post-incongruent incongruent trial contrast, respectively, in the healthy comparison group. Activity in the amygdala was then regressed against the product of the ventral cingulate time-course and the vector of the psychological variable of interest, with the physiological and the psychological variables serving as regressors of no interest, along with the six motion parameters. The results were then analyzed using ANOVAs in SPSS as above.

Small-volume corrections were conducted for the a priori specified ventral cingulate and amygdala regions of interest (48) (p<0.05, family-wise error-corrected) using anatomically-defined masks. The ventral cingulate region of interest was drawn along the contours defined by a recent DTI connectivity parcellation of the cingulate (regions 1 and 2 in (49)), thus significantly expanding upon our previous cingulate mask (23) to include the entire ventral cingulate (21,320 mm³). The amygdala region of interest corresponded to the bilateral amygdala in WFU PickAtlas (left: 1264 mm³; right: 1288 mm³ (50). Results are displayed within these regions of interest only. For the correlation of reaction times with brain activation in the depression only group, we applied a whole-brain correction for the false discovery rate (q<0.05).

### Results

### Behavior

The patient and comparison groups were well matched for age, gender, handedness, and education (see Table 1). As outlined above, we analyzed our data in two parallel ways, with either depression and anxiety as independent and interacting factors (“independent factor model”), or with anxiety and depression diagnoses combined into a single “patient” factor (“common disorder model”). Overall accuracies were not significantly different using either model (comparison group=94.2% [SD=4.7]; anxiety only=93.5% [SD=5.6]; depression only=95.9% [SD=3.4]; comorbid=93.8% [4.6]; all patients=94.2% [SD 4.7]). Average reaction times showed no significant effect of anxiety or depression in the independent factor model, but did show an overall effect of patient status in the common disorders model (F(1,85)=6.4, p<0.05, partial eta squared=0.07; comparison group=766 ms [SD=106]; anxiety only=865 ms [SD=235]; depression only=900 ms [SD=226]; comorbid=861 ms [SD=229]; all patients=872 ms [SD=227]).

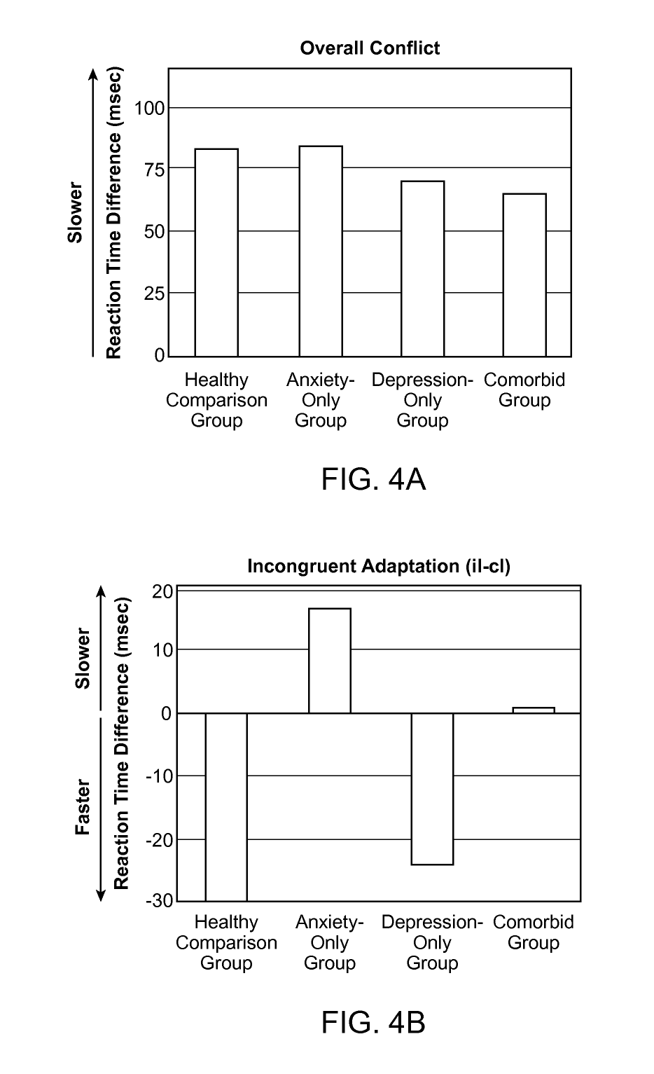

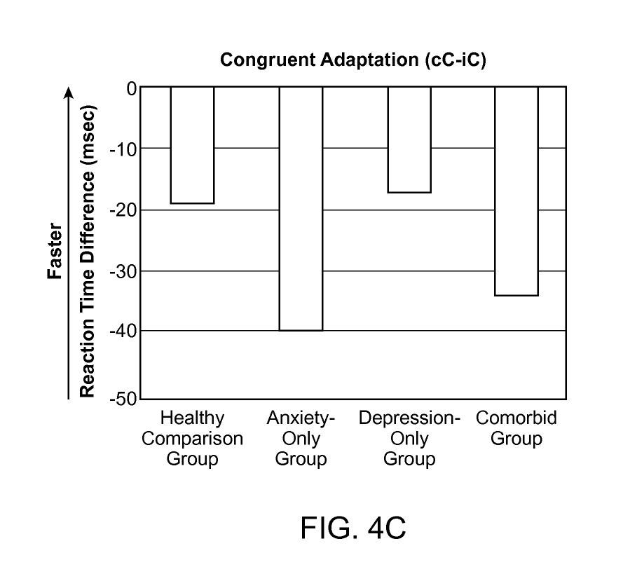

Emotional conflict, as expected, induced a reaction-time slowdown in all groups (see FIG. 4A). There were no significant differences in this slowdown as a function of factors anxiety, depression or patient status. By contrast, there was a significant deficit in emotional conflict adaptation during incongruent trials both as a function of anxiety in the independent factor model (anxiety only and comorbids versus controls and depression only; F(1,85)=8.1, p<0.01, partial eta squared=0.087) and as an effect of patient status in the common disorder model (F(1,85)=4.8, p<0.05, partial eta squared=0.053; see FIG. 4B). The effect of anxiety, but not patient status, remained significant even after controlling for individuals' average reaction times or individuals' scores on scales of anxiety, depression or worry (p<0.01), indicating a categorical effect of diagnosis rather than dimensional effect of anxiety or depression.

Furthermore, consistent with the independent factor model, but not the common disorder model, adaptation during incongruent trials was significantly different between the depression only group and the combination of generalized anxiety disorder only and comorbid groups (F(1,55)=4.3, p<0.05, partial eta squared=0.073). Importantly, this effect remained significant after controlling for either average reaction times or individuals' scores on scales of anxiety, depression or worry (p<0.05). Finally, there were no significant group effects in either the independent factor or common disorder models on adaptation during congruent trials (see FIG. 4C), wherein reaction times are faster for post-congruent congruent trials than post-incongruent congruent trials. Moreover, all individual groups' one-sample t-tests were significant for this measure, suggesting that all the groups showed adaptation during congruent trials, which indicates the specificity of the finding of deficits in adaptation during incongruent trials.

FIGS. 4A-C: Failure to adapt to emotional conflict in anxiety. (A) Reaction time difference scores reflecting the overall effect of emotional conflict (incongruent minus congruent trials), showing no difference between groups. (B) Facilitation in reaction times during emotional conflict adaptation (post-incongruent incongruent trials (iI) faster than post-congruent incongruent trials (cI), resulting in negative reaction time difference scores), showing a deficit as a function of anxiety in the independent factor model (i.e. in the generalized anxiety disorder-only and comorbid groups). (C) Adaptation on congruent trials (post-congruent congruent trials (cC) faster than post-incongruent congruent trials (iC)), showing no group differences.

### Ventral Cingulate-Amygdalar Abnormalities Across Anxiety and Depression

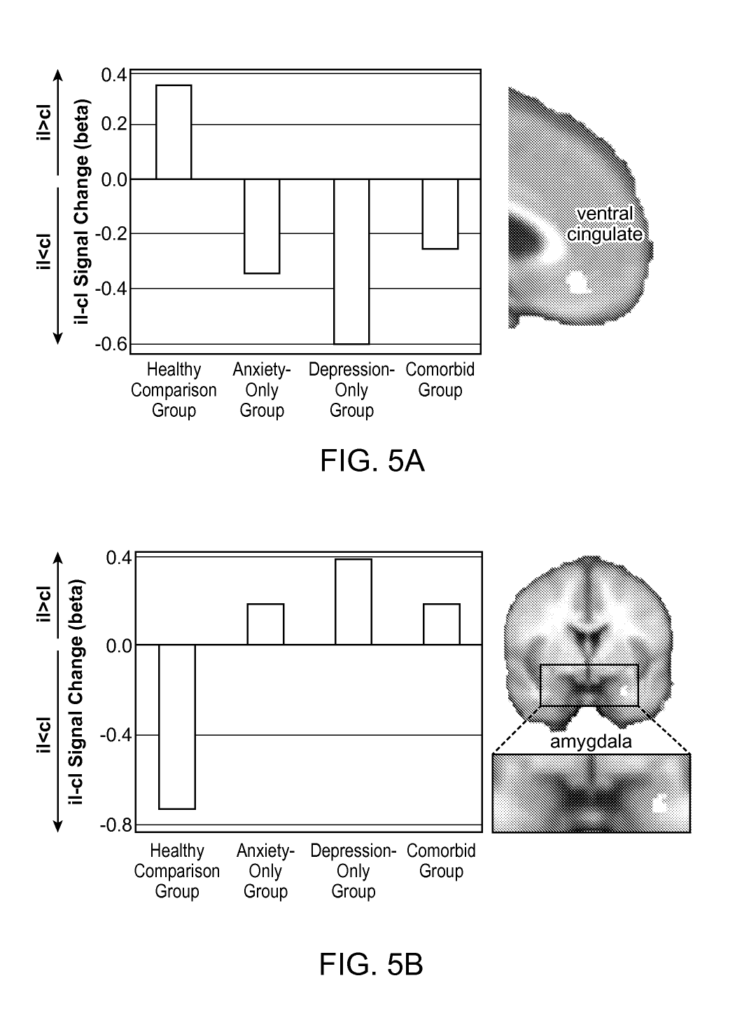

Next, we examined brain activation in the critical contrast examining emotional conflict adaptation during incongruent trials (post-incongruent incongruent trials minus post-congruent incongruent trials), focusing on our ventral cingulate and amygdalar a priori regions of interest. As discussed above, adaptation to emotional conflict is associated with increased activity in the ventral cingulate (post-incongruent incongruent trials>post-congruent incongruent trials) and decreased activation in the amygdala (post-incongruent incongruent trials<post-congruent incongruent trials)—functions attributed to regulation and evaluation of emotional conflict, respectively (21-23). In both regions, we found significant small volume-corrected group differences as a function of patient status in the common disorder model, and no significant effects of anxiety or depression in the independent factor model (see FIGS. 5A and 5B).

The group difference cluster in the ventral cingulate (x=−10, y=28, z=−2; z=4.09 and x=−4, y=40, z=−16; z=3.81, 1008 mm³; partial eta squared=0.123; see mean cluster signal change for each group in FIG. 5A) was driven by significant conflict regulation-related activity increase in the comparison group (t(31)=3.15, p<0.005, d=0.55) and the opposite effect in patients (t(56)=2.59, p=0.01, d=0.34). The group difference cluster in the amygdala (x=28, y=0, z=−28; z=3.25; 160 mm³; partial eta squared=0.092; see cluster means in FIG. 5B), as expected, involved a significant decrease in signal during adaptation in the comparison group (t(31)=2.19, p<0.05, d=0.39), whereas in patients no difference was observed (t(56)=1.5, p>0.1).

FIGS. 5A and 5B: Inability of all patients to activate the ventral cingulate and dampen amygdalar activity during emotional conflict adaptation. Healthy comparison>all patient contrast for the post-incongruent incongruent trial (iI) minus post-congruent incongruent trial (cI) difference within the ventral cingulate (A) and amygdala (B) regions of interest, with a bar graph representing each group's data for this contrast extracted for the cluster shown.

Further breakdown by individual trial types for both the ventral cingulate and amygdala group difference clusters can be found in supplemental FIGS. 3A and 3B, respectively. No group differences were observed in either the ventral cingulate or amygdala for the contrast of post-congruent congruent trials minus post-incongruent congruent trials, or when combining across all trial types, indicating that the group differences during adaptation to emotional conflict did not simply reflect generic consequences of reaction time speedup or task-independent deactivations, respectively. The observed group differences for brain activation and behavior were also unaltered if subjects with comorbid obsessive-compulsive disorder were removed or if patients with comorbid dysthymia in the anxiety-only group were removed.

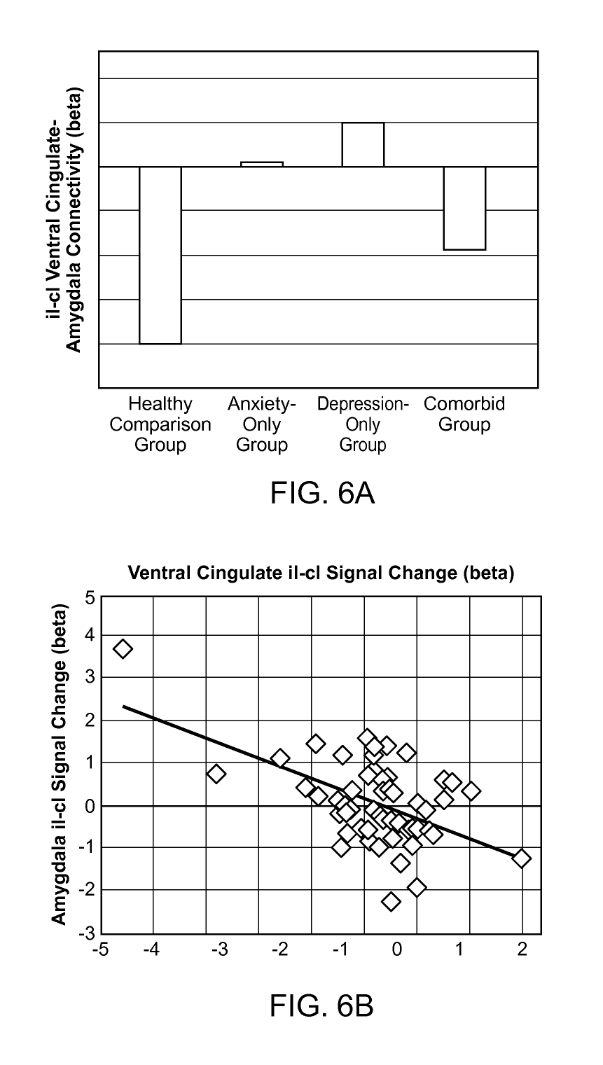

Next, we examined functional connectivity between the ventral cingulate and amygdala using psychophysiological interaction analyses, and found a blunting of the normally negative functional connectivity between these regions across all patients in the common disorders model (F(1,85)=4.4, p<0.05, partial eta squared=0.049; see FIG. 6A). Finally, within the entire patient cohort, we correlated mean incongruent trial adaptation signal for the ventral cingulate and amygdala clusters and found that they were strongly negatively correlated (r=−0.52, p<0.001; see FIG. 6B), consistent with a negative regulatory relationship between these regions in patients, even in the context of an overall deficit in their activation. Robust regression confirmed that this relationship was independent of outliers (p<0.0005). In summary, these data demonstrate a broad deficit in cingulate-amygdalar activation and connectivity during adaptation in all patient cohorts, which is consistent with the behavioral adaptation deficits in the anxiety only and comorbid groups, but does not account for the adaptation seen in the depression only group.

FIGS. 6A and 6B: Ventral cingulate-amygdala functional connectivity and activation correlations. (A) The normally negative post-incongruent incongruent trial (iI) minus post-congruent incongruent trial (cI) functional connectivity between the ventral cingulate and amygdala using psychophysiological interactions is blunted across all patient groups, compared to healthy comparison subjects. (B) Negative correlation between iI-cI activation differences in the ventral cingulate and the amygdala, showing that greater ventral cingulate activity was associated with less amygdala activity even in the context of overall activation abnormalities.

### Compensatory Recruitment of Lateral Anterior Prefrontal Regions in Depression

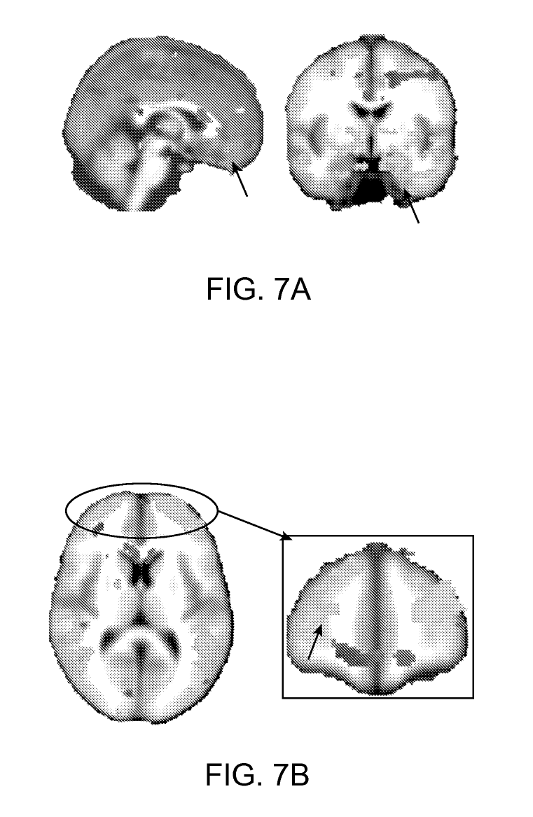

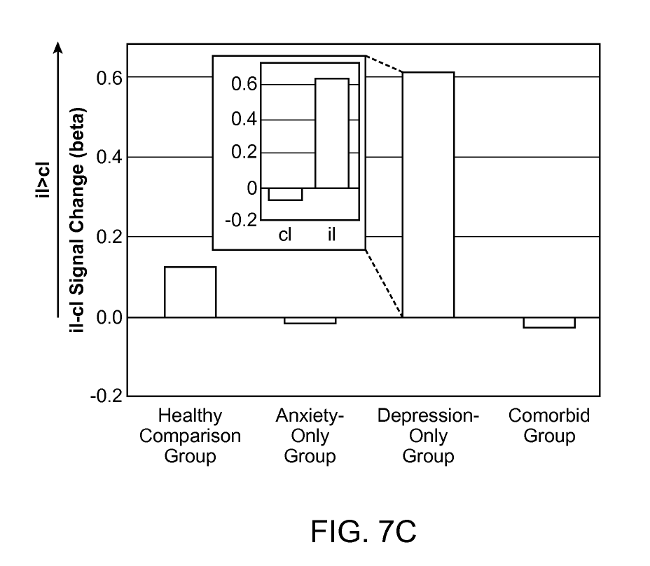

To identify brain regions which may account for the ability of depressed patients to adapt to emotional conflict, we correlated individuals' reaction time difference scores during incongruent trial adaptation (post-incongruent incongruent trials minus post-congruent incongruent trials) with brain activation in the same contrast within the depression only group. Results after whole-brain voxelwise correction for multiple comparisons using the false discovery rate (q<0.05) can be seen in FIG. 7. We found that better incongruent trial conflict adaptation (more negative reaction time difference scores) was associated with progressively less activation in the ventral cingulate (i.e. positive correlation), as well as a greater failure to dampen amygdala activation (i.e. negative correlation; see arrows in FIG. 7A). Thus, though subjects were better able to adapt to emotional conflict, this was associated with a more dysfunctional pattern of activation in the regions associated with emotional conflict adaptation in healthy subjects. By contrast, in the combined generalized anxiety disorder-only and comorbid groups, using a small-volume correction for the amygdala, we found the opposite (and predicted) brain-behavior relationship, wherein better adaptation was associated with greater dampening of the amygdala (x=18, y=2, z=−16; z=3.53; 128 mm³), indicating that the depression-only group is able to adapt by activating a different neural system.

We found three clusters in the frontal lobe, however, in which greater activation in the depression-only group was associated with improved conflict adaptation (i.e. negative correlation; see FIG. 7B). These clusters were located in the left superior (x=−22, y=44, z=44; z=4.89, 3776 mm³) and middle (x=−22, y=48, z=12; z=4.83, 3352 mm³) frontal gyri, and in the right middle frontal gyms (x=30, y=62, z=14; z=4.45, 8080 mm³). These correlations were verified to be independent of outliers and remained similarly significant using robust regression (p<0.001 for all).

We next compared mean activation in each of these clusters (in the post-incongruent incongruent trials minus post-congruent incongruent trials contrast) between the depression only group and the combination of the generalized anxiety disorder only and comorbid groups, which failed to adapt to emotional conflict. Of the three frontal clusters, only activation in the left anterior middle frontal gyms significantly differed between the groups (F(1,55)=7.08, p=0.01, partial eta squared 0.114; see FIG. 7C), which also remained significant after controlling for individuals' scores on scales of anxiety, depression or worry (p<0.005). The inset in FIG. 7C shows the breakdown by trial types of activity in this cluster for the depression only group, with the breakdown for the other groups shown in supplemental FIG. 7. Finally, we conducted a mediation analysis to determine whether activation in the left anterior middle gyms cluster statistically mediated the relationship between group (depression only versus generalized anxiety disorder only and comorbids) and ability to adapt to emotional conflict (reaction time difference scores). A significant mediation relationship existed for this cluster (a (predictor-mediator path)=0.32, p<0.005; b (mediator-criterion variable path)=−28, p<0.001; ab (mediation effect)=−8.9, p<0.05), which also remained significant after controlling for individuals' scores on scales of anxiety, depression or worry (p<0.05 for all).

FIGS. 7A-C: Engagement of compensatory activation in the anterior lateral prefrontal cortices in the depression only group is associated with successful adaptation to emotional conflict in this group. Correlation between post-incongruent incongruent trial (iI) minus post-congruent incongruent trial (cI) reaction time difference scores (more negative=more adaptation) and brain activation for the same contrast, displayed at a whole-brain false discovery rate q<0.05 corrected threshold. (A) Positive correlations in the ventral cingulate and negative correlations in the amygdala (arrows) suggest a greater deficit in these regions when depression only subjects show better reaction time adaptation. (B) Negative correlations in the anterior lateral prefrontal cortex suggest regulation-related recruitment of this region with improved adaptation. (C) Activity for the left anterior middle frontal gyms cluster (arrow in panel B) extracted for the iI-cI contrast for each group, as well as separately for the iI and cI trials (see inset) for the depression only group. These data show that this cluster is only activated in the depression only group, and that this is driven by increased activity in iI trials.

### REFERENCES

1. Mennin D S, Heimberg R G, Fresco D M, Ritter M R. Is generalized anxiety disorder an anxiety or mood disorder? Considering multiple factors as we ponder the fate of GAD. Depress Anxiety. 2008; 25(4):289-299.
2. Watson D. Rethinking the mood and anxiety disorders: a quantitative hierarchical model for DSM-V. J Abnorm Psychol. 2005 November; 114(4):522-536.
3. Goldberg D. Towards DSM-V: the relationship between generalized anxiety disorder and major depressive episode. Psychol Med. 2008 November; 38(11):1671-1675.
4. Craske M G, Rauch S L, Ursano R, Prenoveau J, Pine D S, Zinbarg R E. What is an anxiety disorder? Depress Anxiety. 2009; 26(12):1066-1085.
5. Hettema J M. The nosologic relationship between generalized anxiety disorder and major depression. Depress Anxiety. 2008; 25(4):300-316.
6. Krueger R F. The structure of common mental disorders. Arch Gen Psychiatry. 1999 October; 56(10):921-926.
7. Vollebergh W A, Iedema J, Bijl R V, de Graaf R, Smit F, Ormel J. The structure and stability of common mental disorders: the NEMESIS study. Arch Gen Psychiatry. 2001 June; 58(6):597-603.
8. Clark L A, Watson D. Tripartite model of anxiety and depression: psychometric evidence and taxonomic implications. J Abnorm Psychol. 1991 August; 100(3):316-336.
9. Kendler K S, Neale M C, Kessler R C, Heath A C, Eaves L J. Major depression and generalized anxiety disorder. Same genes, (partly) different environments? Arch Gen Psychiatry. 1992 September; 49(9):716-722.
10. Roy M A, Neale M C, Pedersen N L, Mathe A A, Kendler K S. A twin study of generalized anxiety disorder and major depression. Psychol Med. 1995 September; 25(5):1037-1049.
11. Brown T A, Chorpita B F, Barlow D H. Structural relationships among dimensions of the DSM-IV anxiety and mood disorders and dimensions of negative affect, positive affect, and autonomic arousal. J Abnorm Psychol. 1998 May; 107(2):179-192.
12. Finlay-Jones R, Brown G W. types of stressful life event and the onset of anxiety and depressive disorders. Psychol Med. 1981 November; 11(4):803-815.
13. Brown G W. Life events and affective disorder: replications and limitations. Psychosom Med. 1993 May-June; 55(3):248-259.
14. Dalgleish T, Watts F N. Biases of attention and memory in disorders of anxiety and depression. Clinical Psychology Review. [doi: DOI: 10.1016/0272-7358(90)90098-U]. 1990; 10(5):589-604.
15. Mogg K, Bradley B. Attentional Bias in Generalized Anxiety Disorder Versus Depressive Disorder. Cognitive Therapy and Research. 2005; 29(1):29-45.
16. Drevets W C, Price J L, Furey M L. Brain structural and functional abnormalities in mood disorders: implications for neurocircuitry models of depression. Brain Struct Funct. 2008 September; 213(1-2):93-118.
17. Etkin A, Wager T D. Functional neuroimaging of anxiety: a meta-analysis of emotional processing in PTSD, social anxiety disorder, and specific phobia. Am J. Psychiatry. 2007 October; 164(10):1476-1488.
18. Fava M, Rush A J, Alpert J E, Balasubramani G K, Wisniewski S R, Carmin C N, Biggs M M, Zisook S, Leuchter A, Howland R, Warden D, Trivedi M H. Difference in treatment outcome in outpatients with anxious versus nonanxious depression: a STAR*D report. Am J. Psychiatry. 2008 March; 165(3):342-351.
19. Binder E B, Owens M J, Liu W, Deveau T C, Rush A J, Trivedi M H, Fava M, Bradley B, Ressler K J, Nemeroff C B. Association of polymorphisms in genes regulating the corticotropin-releasing factor system with antidepressant treatment response. Arch Gen Psychiatry. 2010 April; 67(4):369-379.
20. Etkin A, Egner T, Kalisch R. Emotional processing in anterior cingulate and medial prefrontal cortex. Trends Cogn Sci. in press December 15.
21. Egner T, Etkin A, Gale S, Hirsch J. Dissociable neural systems resolve conflict from emotional versus nonemotional distracters. Cereb Cortex. 2008 June; 18(6):1475-1484.
22. Etkin A, Egner T, Peraza D M, Kandel E R, Hirsch J. Resolving emotional conflict: a role for the rostral anterior cingulate cortex in modulating activity in the amygdala. Neuron. 2006 Sep. 21; 51(6):871-882.
23. Etkin A, Prater K E, Hoeft F, Menon V, Schatzberg A F. Failure of anterior cingulate activation and connectivity with the amygdala during implicit regulation of emotional processing in generalized anxiety disorder. Am J. Psychiatry. 2010 May; 167(5):545-554.
24. Botvinick M, Nystrom L E, Fissell K, Carter C S, Cohen J D. Conflict monitoring versus selection-for-action in anterior cingulate cortex. Nature. 1999 Nov. 11; 402(6758):179-181.
25. Egner T, Hirsch J. Cognitive control mechanisms resolve conflict through cortical amplification of task-relevant information. Nat Neurosci. 2005 December; 8(12):1784-1790.
26. Gratton G, Coles M G, Donchin E. Optimizing the use of information: strategic control of activation of responses. J Exp Psychol Gen. 1992 December; 121(4):480-506.
27. Kerns J G, Cohen J D, MacDonald A W, 3rd, Cho R Y, Stenger V A, Carter C S. Anterior cingulate conflict monitoring and adjustments in control. Science. 2004 Feb. 13; 303(5660):1023-1026.
28. Botvinick M M, Braver T S, Barch D M, Carter C S, Cohen J D. Conflict monitoring and cognitive control. Psychol Rev. 2001 July; 108(3):624-652.
29. Botvinick M M, Cohen J D, Carter C S. Conflict monitoring and anterior cingulate cortex: an update. Trends Cogn Sci. 2004 December; 8(12):539-546.
30. Mansouri F A, Tanaka K, Buckley M J. Conflict-induced behavioural adjustment: a clue to the executive functions of the prefrontal cortex. Nat Rev Neurosci. 2009 February; 10(2):141-152.
31. Carter C S, Macdonald A M, Botvinick M, Ross L L, Stenger V A, Noll D, Cohen J D.

Parsing executive processes: strategic vs. evaluative functions of the anterior cingulate cortex. Proc Natl Acad Sci USA. 2000 Feb. 15; 97(4):1944-1948.
32. Egner T, Hirsch J. The neural correlates and functional integration of cognitive control in a Stroop task. Neuroimage. 2005 Jan. 15; 24(2):539-547.
33. APA. Diagnostic and statistical manual of mental disorders. 4th ed. Washington D.C.: American Psychiatric Press; 1994.
34. Sheehan D V, Lecrubier Y, Sheehan K H, Amorim P, Janays J, Weiller E, Hergueta T, Baker R, Dunbar G C. The Mini-International Neuropsychiatric Interview (M.I.N.I.): the development and validation of a structured diagnostic psychiatric interview for DSM-IV and ICD-10. J Clin Psychiatry. 1998; 59 Suppl 20:22-33; quiz 34-57.
35. Sheehan D V, Lecrubier Y, Harnett-Sheehan K, Janays J, Weiller E, Bonora L I, Keskiner A, Schinka J, Knapp E, Sheehan M F, Dunbar G C. Reliability and Validity of the MINI

International Neuropsychiatric Interview (M.I.N.I.): According to the SCID-P. European Psychiatry. 1997; 12:232-241.
36. Spielberger C D, Gorsuch R L, Lushene R E. Manual for the State-Trait Anxiety Inventory. Palo Alto, Calif.: Consulting Psychologists Press; 1970.
37. Meyer T J, Miller M L, Metzger R L, Borkovec T D. Development and validation of the Penn State Worry Questionnaire. Behav Res Ther. 1990; 28(6):487-495.
38. Beck A T, Steer R A. Beck Anxiety Inventory Manual. San Antonio, Tex.: Psychological Corporation; 1993.
39. Beck A T, Steer R A, Brown G K. Manual for Beck Depression Inventory II (BDI-II). San Antonio, Tex.: Psychological Corporation; 1996.
40. Watson D, Clark L A, Weber K, Assenheimer J S, Strauss M E, McCormick R A. Testing a tripartite model: II. Exploring the symptom structure of anxiety and depression in student, adult, and patient samples. J Abnorm Psychol. 1995 February; 104(1):15-25.
41. Watson D, Weber K, Assenheimer J S, Clark L A, Strauss M E, McCormick R A. Testing a tripartite model: I. Evaluating the convergent and discriminant validity of anxiety and depression symptom scales. J Abnorm Psychol. 1995 February; 104(1):3-14.
42. Ekman P, Friesen W V. Pictures of Facial Affect. Palo Alto, Calif.: Consulting Psychologists; 1976.
43. Glover G H, Lai S. Self-navigated spiral fMRI: interleaved versus single-shot. Magn Reson Med. 1998 March; 39(3):361-368.
44. Friston K J, Ashbumer J, Poline J B, Frith C D, Heather J D, Frackowiak R S. Spatial registration and normalization of images. Hum Brain Mapp. 1995; 2:165-189.
45. Macey P M, Macey K E, Kumar R, Harper R M. A method for removal of global effects from fMRI time series. Neuroimage. 2004 May; 22(1):360-366.
46. Friston K J, Holmes A P, Worsley K J, Poline J B, Frith C D, Frackowiak R S. Statistical parametric maps in functional imaging: a general linear approach. Hum Brain Mapp. 1995; 2:189-210.
47. Friston K J, Buechel C, Fink G R, Morris J, Rolls E, Dolan R J. Psychophysiological and modulatory interactions in neuroimaging. Neuroimage. 1997 October; 6(3):218-229.
48. Worsley K J, Marren S, Neelin P, Vandal A C, Friston K J, Evans A C. A unified statistical approach for determining significant signals in images of cerebral activation. Hum Brain Mapp. 1996; 4(1):58-73.
49. Beckmann M, Johansen-Berg H, Rushworth M F. Connectivity-based parcellation of human cingulate cortex and its relation to functional specialization. J. Neurosci. 2009 Jan. 28; 29(4):1175-1190.
50. Maldjian J A, Laurienti P J, Kraft R A, Burdette J H. An automated method for neuroanatomic and cytoarchitectonic atlas-based interrogation of fMRI data sets. Neuroimage. 2003 July; 19(3):1233-1239.
51. Beesdo K, Lau J Y, Guyer A E, McClure-Tone E B, Monk C S, Nelson E E, Fromm S J, Goldwin M A, Wittchen H U, Leibenluft E, Ernst M, Pine D S. Common and distinct amygdala-function perturbations in depressed vs anxious adolescents. Arch Gen Psychiatry. 2009 March; 66(3):275-285.
52. Thomas K M, Drevets W C, Dahl R E, Ryan N D, Birmaher B, Eccard C H, Axelson D, Whalen P J, Casey B J. Amygdala response to fearful faces in anxious and depressed children. Arch Gen Psychiatry. 2001 November; 58(11):1057-1063.
53. Inset T, Cuthbert B, Garvey M, Heinssen R, Pine D S, Quinn K, Sanislow C, Wang P. Research domain criteria (RDoC): toward a new classification framework for research on mental disorders. Am J. Psychiatry. 2010 July; 167(7):748-751.
54. Uher R, McGuffin P. The moderation by the serotonin transporter gene of environmental adversity in the etiology of depression: 2009 update. Mol Psychiatry. 2010 January; 15(1):18-22.
55. Lesch K P, Bengel D, Heils A, Sabot S Z, Greenberg B D, Petri S, Benjamin J, Muller C R, Hamer D H, Murphy D L. Association of anxiety-related traits with a polymorphism in the serotonin transporter gene regulatory region. Science. 1996 Nov. 29; 274(5292):1527-1531.
56. Roiser J P, de Martino B, Tan G C, Kumaran D, Seymour B, Wood N W, Dolan R J. A genetically mediated bias in decision making driven by failure of amygdala control. J. Neurosci. 2009 May 6; 29(18):5985-5991.
57. Munafo M R, Brown S M, Hariri A R. Serotonin transporter (5-HTTLPR) genotype and amygdala activation: a meta-analysis. Biol Psychiatry. 2008 May 1; 63(9):852-857.
58. Pezawas L, Meyer-Lindenberg A, Drabant E M, Verchinski B A, Munoz K E, Kolachana B S, Egan M F, Mattay V S, Hariri A R, Weinberger D R. 5-HTTLPR polymorphism impacts human cingulate-amygdala interactions: a genetic susceptibility mechanism for depression. Nat Neurosci. 2005 June; 8(6):828-834.
59. Scharinger C, Rabl U, Sitte H H, Pezawas L. Imaging genetics of mood disorders. Neuroimage. in press February 13.
60. Meyer-Lindenberg A, Buckholtz J W, Kolachana B, A R H, Pezawas L, Blasi G, Wabnitz A, Honea R, Verchinski B, Callicott J H, Egan M, Mattay V, Weinberger D R. Neural mechanisms of genetic risk for impulsivity and violence in humans. Proc Natl Acad Sci USA. 2006 Apr. 18; 103(16):6269-6274.
61. Canli T, Congdon E, Gutknecht L, Constable R T, Lesch K P. Amygdala responsiveness is modulated by tryptophan hydroxylase-2 gene variation. J Neural Transm. 2005 November; 112(11):1479-1485.
62. Soliman F, Glatt C E, Bath K G, Levita L, Jones R M, Pattwell S S, Jing D, Tottenham N, Amso D, Somerville L H, Voss H U, Glover G, Ballon D J, Liston C, Teslovich T, Van Kempen T, Lee F S, Casey B J. A genetic variant BDNF polymorphism alters extinction learning in both mouse and human. Science. 2010 Feb. 12; 327(5967):863-866.
63. Monk C S, Klein R G, Telzer E H, Schroth E A, Mannuzza S, Moulton J L, 3rd, Guardino M, Masten C L, McClure-Tone E B, Fromm S, Blair R J, Pine D S, Ernst M. Amygdala and nucleus accumbens activation to emotional facial expressions in children and adolescents at risk for major depression. Am J. Psychiatry. 2008 January; 165(1):90-98.
64. Mannie Z N, Norbury R, Murphy S E, Inkster B, Harmer C J, Cowen P J. Affective modulation of anterior cingulate cortex in young people at increased familial risk of depression. Br J. Psychiatry. 2008 May; 192(5):356-361.
65. van Tol M J, van der Wee N J, van den Heuvel O A, Nielen M M, Demenescu L R, Aleman A, Renken R, van Buchem M A, Zitman F G, Veltman D J. Regional brain volume in depression and anxiety disorders. Arch Gen Psychiatry. 2010 October; 67(10):1002-1011.
66. Gilbert S J, Gonen-Yaacovi G, Benoit R G, Voile E, Burgess P W. Distinct functional connectivity associated with lateral versus medial rostral prefrontal cortex: A meta-analysis. Neuroimage. 2010 Jul. 21.
67. Etkin A, Prater K E, Schatzberg A F, Menon V, Greicius M D. Disrupted amygdalar subregion functional connectivity and evidence of a compensatory network in generalized anxiety disorder. Arch Gen Psychiatry. 2009 December; 66(12):1361-1372.

### CONCLUSION

The foregoing description has been presented for purposes of illustration and description. It is not intended to be exhaustive or to limit the invention to the precise form disclosed. Other modifications and variations may be possible in light of the above teachings. The embodiments were chosen and described in order to best explain the principles of the invention and its practical application, and to thereby enable others skilled in the art to best utilize the invention in various embodiments and various modifications as are suited to the particular use contemplated. It is intended that the appended claims be construed to include other alternative embodiments of the invention; including equivalent structures, components, methods, and means.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It is to be appreciated that the Detailed Description section, and not the Summary and Abstract sections, is intended to be used to interpret the claims. The Summary and Abstract sections may set forth one or more, but not all exemplary embodiments of the present invention as contemplated by the inventor(s), and thus, are not intended to limit the present invention and the appended claims in any way.

What is claimed is:

1. A computer-readable storage medium used for treating a patient with, or at risk of, a psychiatric disorder by training the patient's implicit emotional regulation, comprising:
instructions executable by at least one processing device that, when executed, cause the processing device to
(a) present an emotional conflict task to the patient, wherein the emotional conflict task includes presenting a series of stimuli-pairs, each stimuli-pair representing at least one emotional significance,
(b) receive an input from the patient in response to the stimuli-pair,
(c) measure a time between when the patient is presented with the stimuli-pair and when the input is received from the patient, and
(d) modify how a subsequent stimuli-pair is presented to the patient based on the amount of time between when the patient is presented with the stimuli-pair and when the input is received from the patient.

2. The computer-readable storage medium of claim 1, further comprising:
instructions executable by at least one processing device that, when executed, cause the processing device to assess the ability of the patient to correctly identify the emotional significance of the stimuli-pair.

3. The computer-readable storage medium of claim 2, further comprising:
instructions executable by at least one processing device that, when executed, cause the processing device to:
measure a time between when the patient is subjected to a stimuli-pair and when the patient correctly identifies the emotional significance of the stimuli-pair; and
modify how a subsequent stimuli-pair is presented to the patient based on the amount of time between when the patient is presented with the stimuli-pair and when the patient correctly identifies the emotional significance of the stimuli-pair.

4. The computer-readable storage medium of claim 1, wherein at least one of the stimuli-pairs includes two incongruent stimuli.

5. The computer-readable storage medium of claim 1, wherein at least one of the stimuli-pairs includes two congruent stimuli.

6. The computer-readable storage medium of claim 1, wherein the series of stimuli-pairs include a sequence of stimuli-pairs selected from the group consisting of: an incongruent stimuli-pair preceded by another incongruent stimuli-pair, an incongruent stimuli-pair preceded by a congruent stimuli-pair, a congruent stimuli-pair preceded by another congruent stimuli-pair, and a congruent stimuli-pair preceded by an incongruent stimuli-pair.

7. The computer-readable storage medium of claim 1, wherein at least one stimuli-pair includes an image and an overlaying word.

8. The computer-readable storage medium of claim 1, wherein at least one stimuli-pair includes two images.

9. The computer-readable storage medium of claim 1, wherein at least one a stimuli-pair includes an emotional face with an emotional label overlaying the emotional face.

10. The computer-readable storage medium of claim 1, wherein the emotional conflict task includes a series of fixation crosses displayed between stimuli-pairs.

11. The computer-readable storage medium of claim 10, further comprising:
instructions executable by at least one processing device that, when executed, cause the processing device to change the amount of time in which a fixation cross is presented to the patient based on how long the patient has used the computer-readable storage medium.

12. The computer-readable storage medium of claim 1, wherein the patient is subject to the emotional conflict task for less than 10 minutes.

13. The computer-readable storage medium of claim 1, wherein the patient is subject to the emotional conflict task for less than 4 minutes.

14. The computer-readable storage medium of claim 1, wherein the emotional significance is selected from the group consisting of: happiness, disgust, fear, sadness, surprise, anger, an idiosyncratic emotion, and equivalents thereof.

15. A computer-readable storage medium used for treating a patient with, or at risk of, a psychiatric disorder by training the patient's implicit emotional regulation, comprising:
instructions executable by at least one processing device that, when executed, cause the processing device to
(a) present an emotional conflict task to the patient, wherein the emotional conflict task includes presenting a series of stimuli-pairs, each stimuli-pair representing at least one emotional significance,
(b) receive an input from the patient in response to the stimuli-pair,
(c) assess the ability of the patient to correctly identify the emotional significance of the stimuli-pair, and
(d) modify how a subsequent stimuli-pair is presented to the patient based on whether the patient correctly identified the emotional significance of a preceding stimuli-pair.

16. The computer-readable storage medium of claim 15, wherein if the patient fails to correctly identify the emotional significance of a preceding stimuli-pair, a subsequent stimuli-pair is presented to the patient for a length of time between 63-73 milliseconds (ms) greater than the length of time that the preceding stimuli-pair was presented to the patient.

17. The computer-readable storage medium of claim 15, wherein if the patient correctly identifies the emotional significance of a preceding stimuli-pair, a subsequent stimuli-pair is presented to the patient for a length of time between 46-56 ms greater than the length of time that the preceding stimuli-pair was presented to the patient.

18. The computer-readable storage medium of claim 15, wherein if the patient correctly identifies the emotional significance of two preceding stimuli-pairs, a subsequent stimuli-pair is presented to the patient for a length of time between 29-39 ms greater than the length of time that the preceding stimuli-pair was presented to the patient.

19. The computer-readable storage medium of claim 15, wherein if the patient correctly identifies the emotional significance of three preceding stimuli-pairs, a subsequent stimuli-pair is presented to the patient for a length of time between 12-22 ms greater than the length of time that the preceding stimuli-pair was presented to the patient.

20. The computer-readable storage medium of claim 15, wherein if the patient correctly identifies the emotional significance of four preceding stimuli-pairs, a subsequent stimuli-pair is presented to the patient for a length of time about equal to the length of time that the preceding stimuli-pair was presented to the patient.

21. The computer-readable storage medium of claim 15, wherein if the patient correctly identifies the emotional significance of five preceding stimuli-pairs, a subsequent stimuli-pair is presented to the patient for a length of time between 12-22 ms less than the length of time that the preceding stimuli-pair was presented to the patient.

22. A computerized method for treating a patient with, or at risk of, a psychiatric disorder by training the patient's implicit emotional regulation, the computerized method comprising:
(a) presenting an emotional conflict task to the patient, wherein the emotional conflict task includes presenting a series of stimuli-pairs, each stimuli-pair representing at least one emotional significance;
(b) receiving an input from the patient in response to one of the stimuli-pair;
(c) measuring a time between when the patient is presented with the stimuli-pair and when the input is received from the patient; and
(d) modifying how a subsequent stimuli-pair is presented to the patient based on the amount of time between when the patient is presented with the stimuli-pair and when the input is received from the patient.

23. A computerized method for treating a patient with, or at risk of, a psychiatric disorder by training the patient's implicit emotional regulation, the computerized method comprising:
(a) presenting an emotional conflict task to the patient, wherein the emotional conflict task includes presenting a series of stimuli-pairs, each stimuli-pair representing at least one emotional significance;
(b) receiving an input from the patient in response to one of the stimuli-pair;
(c) assessing the ability of the patient to correctly identify the emotional significance of the stimuli-pair; and
(d) modifying how a subsequent stimuli-pair is presented to the patient based on whether the patient correctly identified the emotional significance of a preceding stimuli-pair.

24. A method of assessing the effectiveness of a treatment for a psychiatric disorder selected from an anxiety disorder and a depressive disorder in an individual, the method comprising:
assessing the ability of the individual to implicitly regulate emotional conflict by subjecting the individual to an emotional conflict task, wherein the ability of the individual to implicitly regulate emotional conflict provides an indication of the effectiveness of the treatment.

25. The method of claim 24, wherein the treatment is a drug treatment.

26. The method of claim 25, wherein the drug treatment is an experimental drug being tested for efficacy in treating an anxiety disorder.

27. The method of claim 24, further comprising:
detecting activation in limbic and prefrontal brain regions in the individual, wherein the activation, if any, provides an indication of the effectiveness of the treatment.

28. The method of claim 24, wherein said assessing comprises determining a reaction time adaptation to emotionally incongruent trials of the emotional conflict task.

29. The method of claim 25, wherein the drug treatment comprises administration of a drug selected from a selective serotonin reuptake inhibitor, a serotonin and norepinephrine reuptake inhibitor, a dopamine reuptake inhibitor, a tetracyclic antidepressant, a combined reuptake inhibitor, a receptor blocker, tricyclic antidepressant, a monoamine oxidase inhibitor, a benzodiazepine, a beta-blocker, and a non-benzodiazepine hypnotic.

30. A method of predicting response of an individual to treatment for a psychiatric disorder selected from an anxiety disorder and a depressive disorder, the method comprising:
assessing the ability of the individual to implicitly regulate emotional conflict by subjecting the individual to an emotional conflict task,
wherein failure to implicitly regulate emotional conflict indicates a reduced likelihood that the individual will exhibit a beneficial clinical response to treatment for an anxiety disorder, and
wherein ability to implicitly regulate emotional conflict indicates an increased likelihood that the individual will exhibit a beneficial clinical response to treatment for a depressive disorder.
